(19) United States
(12) Patent Application Publication
(10) Pub. No.: US 2024/0197594 A1
(43) Pub. Date: Jun. 20, 2024

(54) PERSONAL CARE COMPOSITIONS

(71) Applicant: Colgate-Palmolive Company, New York, NY (US)

(72) Inventors: Min LI, Bridgewater, NJ (US); Junhong MAO, Plainsboro, NJ (US); Thomas BOYD, Metuchen, NJ (US)

(73) Assignee: Colgate-Palmolive Company, New York, NY (US)

(21) Appl. No.: 18/544,750

(22) Filed: Dec. 19, 2023

Related U.S. Application Data
(60) Provisional application No. 63/476,002, filed on Dec. 19, 2022.

(51) Int. Cl. A61K 8/365; A61K 8/44; A61Q 19/08

(52) U.S. Cl. CPC A61K 8/365; A61K 8/44; A61Q 19/08; A61K 2800/5922

(57) ABSTRACT

Described herein are post-biotic compositions and personal care compositions thereof for rebalancing skin microbiome and personal care compositions containing the same. In accordance with an aspect of the invention, provided is a post-biotic composition comprising a short chain fatty acid selected from lactic acid, salt thereof and a combination thereof; and a plurality of amino acids, the plurality of amino acids comprising at least 2 amino acids selected from histidine, glycine, threonine, arginine, alanine, valine, proline, a salt thereof, and a combination of two or more thereof.

### CROSS-REFERENCE TO RELATED APPLICATIONS

This application claims the benefit of priority from U.S. Provisional Application No. 63/476,002, filed Dec. 19, 2022, the contents of which are hereby incorporated herein by reference in their entirety.

### BACKGROUND

Skin is the body's first line of defense against infections and environmental stressors. It acts as a major physical and immunological protective barrier, but also plays a critical role in temperature regulation, water holding, vitamin D production, and sensing. Its outermost surface consists of a lipid- and protein-laden cornified layer dotted with hair follicles and eccrine glands that secrete lipids, antimicrobial peptides (AMPs), enzymes, salts, etc. It harbors microbial communities living in a range of physiologically and anatomically distinct niches. Overall, this constitutes a highly heterogeneous and complex system.

During adulthood, absent any specific skin condition, the skin microbiome remains relatively stable, despite the large inter-individual variability, suggesting that mutualistic and commensal interactions exist among microbes and between microbes and host, even for bacterial species often considered as opportunistic pathogens. Under healthy skin conditions, most of the microbes living on the skin behave as commensal or mutualistic organisms. Through various mechanisms, such as the stimulation of innate factor secretion or antimicrobial peptides (AMPs), they maintain the microflora composition avoiding the spread of opportunistic parasites, while also contributing to the education of the immune system and to healthy skin barrier homeostasis. In case of barrier breach or immunosuppression, these carefully balanced relationships may transition from commensalism to pathogenicity, a transition referred to as dysbiosis, enabling the overgrowth of pathogenic species, common in skin conditions such as acne, psoriasis, ulcer, and atopic dermatitis.

Probiotics are known to have beneficial effects on skin microflora balance. In particular, it is desirable to inhibit harmful bacteria, while promoting the growth of beneficial bacteria. However, the ability to provide this dual benefit still remains a challenge.

In addition, skin tends to lose its elasticity and/or firmness as it ages. Current options for maintaining skin elasticity and firmness are sub-optimal.

As such, embodiments of the present invention are designed to provide these, and other, benefits.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The present disclosure relates to post-biotic compositions and personal care compositions thereof for rebalancing skin microbiome and personal care compositions containing the same. In accordance with an aspect of the invention, provided is a post-biotic composition comprising a short chain fatty acid selected from lactic acid, salt thereof and a combination thereof; and a plurality of amino acids, the plurality of amino acids comprising at least 2 amino acids selected from histidine, glycine, threonine, arginine, alanine, valine, proline, a salt thereof, and a combination of two or more thereof.

In accordance with another aspect, provided is a personal care composition comprising a polyol; optionally, a fatty compound; optionally, a thickener; and a post-biotic system comprising: (a) a short chain fatty acid blend comprising (i) a first short chain fatty acid selected from lactic acid, salt thereof and a combination thereof, and (ii) a second short chain fatty acid selected from acetic acid, pyruvic acid, a salt thereof, and a combination of two or more thereof, and (b) a plurality of amino acids, the plurality of amino acids comprising at least 2 amino acids selected from histidine, glycine, threonine, arginine, alanine, valine, proline, a salt thereof, and a combination of two or more thereof.

### BRIEF DESCRIPTION OF THE DRAWINGS

The present invention will become more fully understood from the detailed description and the accompanying drawings, in which:

FIG. 1 is a bar graph illustrating the effect of non-limiting, exemplary personal care compositions and a comparative composition on the growth of E. coli, C. striatum, and S. aureus, and S. epidermidis in accordance with aspects of the invention.

FIG. 2 is a bar graph illustrating the effect of non-limiting, exemplary personal care compositions and a comparative composition on Filaggrin expression in accordance with aspects of the invention.

FIG. 3 is a bar graph illustrating the effect of exemplary personal care compositions and a comparative composition on total collagen expression in accordance with aspects of the invention.

FIG. 4 is a bar graph illustrating the effect of exemplary personal care compositions and a comparative composition on collagen III expression in accordance with aspects of the invention.

It should be understood that the various aspects of the invention are not limited to the arrangements and instrumentality shown in the drawings.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “hr”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

The phrase “MRS agar” refers to De Man, Rogosa and Sharpe agar, which is a selective culture medium designed to favor the growth of Lactobacillus. The phrase “TSB medium” refers to tryptic soy broth or trypticase soy broth, which is used in microbiology laboratories as a culture broth to grow aerobic bacteria. The phrase “PBS wash” refers to phosphate-buffered saline wash. The abbreviation “qPCR” is quantitative polymerase chain reaction.

“Volatile”, as used herein, means having a flash point of less than about 100° C. “Non-volatile”, as used herein, means having a flash point of greater than about 100° C.

When referring to chemical structures, and names, the symbols “C”, “H”, and “O” mean carbon, hydrogen, and oxygen, respectively. The symbols “-”, “=” and “≡” mean single bond, double bond, and triple bond, respectively.

Any member in a list of species that is used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that defines or exemplifies the genus is open, and it is given that other species may exist that define or exemplify the genus just as well as, or better than, any other species listed.

The phrases, “a mixture thereof,” “a combination thereof,” or “a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the hair cleansing composition includes both components (or the composition includes more than two components that overlap), an overlapping compound does not represent more than one component. For example, certain compounds may be characterized as both an emulsifier and a surfactant. If a particular hair composition includes both an emulsifier and a surfactant, the compounds that may be characterized as both an emulsifier and a surfactant will serve only as either the emulsifier or the surfactant—not both.

For readability purposes, the chemical functional groups are in their adjective form; for each of the adjectives, the word “group” is assumed. For example, the adjective “alkyl” without a noun thereafter, should be read as “an alkyl group.”

Escherichia coli is a Gram-negative, facultative anaerobic, rod-shaped, coliform bacterium of the genus Escherichia that is commonly found in the lower intestine of warm-blooded organisms (endotherms). Most E. coli strains are harmless, but some serotypes can cause serious food poisoning in their hosts and are occasionally responsible for product recalls due to food contamination.

Staphylococcus aureus is a Gram-positive, round-shaped bacterium that is a member of the Firmicutes, and it is a usual member of the microbiota of the body, frequently found in the upper respiratory tract and on the skin. It is often positive for catalase and nitrate reduction and is a facultative anaerobe that can grow without the need for oxygen. Skin infections are the most common form of S. aureus infection. This can manifest in various ways, including small benign boils, folliculitis, impetigo, cellulitis, and more severe, invasive soft-tissue infections. S. aureus is extremely prevalent in persons with atopic dermatitis, more commonly known as eczema. It is commonly found in fertile, active places, including the armpits, hair, and scalp. Large pimples that appear in those areas may exacerbate the infection if lacerated.

Corynebacterium striatum is a gram-positive bacterium that assumes a “club-like” morphology, more formally known as a corynebacteria structure. Corynebacterium striatum is generally considered an opportunistic pathogenic that can cause infections in human skin.

Staphylococcus epidermidis, a Gram-positive bacterium, is a part of the normal human flora, typically the skin flora, and less commonly the mucosal flora. It is a facultative anaerobic bacteria.

Aspects of the invention relate to post-biotic compositions for rebalancing skin microbiome. Additional aspects of the invention relate to personal care compositions containing a post-biotic compositions as disclosed herein. When incorporated into a personal care composition, the post-biotic composition is generally referred to as a post-biotic system.

In accordance with an aspect of the invention, provided is a post-biotic composition comprising a short chain fatty acid selected from lactic acid, salt thereof and a combination thereof; and a plurality of amino acids, the plurality of amino acids comprising at least 2 amino acids selected from histidine, glycine, threonine, arginine, alanine, valine, proline, a salt thereof, and a combination of two or more thereof.

In accordance with another aspect, provided is a personal care composition comprising a polyol; optionally, a fatty compound; optionally, a thickener; and a post-biotic system comprising: (a) a short chain fatty acid blend comprising (i) a first short chain fatty acid selected from lactic acid, salt thereof and a combination thereof, and (ii) a second short chain fatty acid selected from acetic acid, pyruvic acid, a salt thereof, and a combination of two or more thereof, and (b) a plurality of amino acids, the plurality of amino acids comprising at least 2 amino acids selected from histidine, glycine, threonine, arginine, alanine, valine, proline, a salt thereof, and a combination of two or more thereof.

The post-biotic compositions and personal care compositions disclosed herein may, preferably, be formulated to inhibit and/or reduce the growth of C. striatum, E. coli, and S. aureus. It was further discovered that certain embodiments can maintain or increase the growth of S. epidermidis in addition to inhibiting and/or reducing the growth of C. striatum, E. coli, and/or S. aureus. For example, the post-biotic compositions and/or personal care compositions may inhibit the growth of C. striatum, E. coli, and/or S. aureus by about 5% or more (cumulatively or individually) and increase the growth of S. epidermidis by about 5% or more by counting CFU/mL after incubating a 2 ml of a bacterial culture having a 0.1 optical density at 610 nm using a UV-VIS Spectrometer for C. striatum, S. aureus, E. coli, and S. epidermidis with a 2 ml of 1% of the post-biotic composition or personal care composition thereof at a temperature of 37° C. for 3 hours. In some embodiment, the post-biotic compositions and/or personal care compositions thereof may inhibit the growth of C. striatum, E. coli, and/or S. aureus, cumulatively or individually, by about 5% or more, about 7% or more, about 10% or more, about 13% or more, about 16% or more, about 19% or more, about 23% or more, about 27% or more, or any range thereof. Additionally or alternatively, certain embodiments of the post-biotic compositions and/or personal care compositions thereof may increase the growth of S. epidermidis by about 5% or more, about 7% or more, about 10% or more, about 13% or more, about 16% or more, about 19% or more, about 23% or more, about 27% or more, or any range thereof.

Preferably, the post-biotic compositions and/or personal care compositions thereof may be formulated to inhibit the growth of C. striatum, E. coli, and S. aureus and increase the growth of S. epidermidis, such that the ratio of S. epidermidis to the combination of C. striatum, E. coli, and S. aureus is about 0.8 or more, based on a log count of C. striatum, E. coli, and S. aureus divided by a log count of S. epidermidis after incubating a 2 ml of a bacterial culture having a 0.1 optical density at 610 nm using a UV-VIS Spectrometer for C. striatum, E. coli, S. aureus, and S. epidermidis with a 2 ml of 1% the post-biotic compositions or personal care compositions thereof at a temperature of 37° C. for 3 hours. For example, the post-biotic compositions and/or personal care compositions thereof may inhibit the growth of C. striatum, E. coli, and S. aureus and increase the growth of S. epidermidis, such that the ratio of S. epidermidis to the combination of C. striatum, E. coli, and S. aureus is about 1 or more, about 1.25 or more, about 1.5 or more, about 1.75 or more, about 2 or more, about 2.3 or more, about 2.6 or more, about 2.9 or more, about 3.1 or more, about 3.4 or more, or any range thereof.

In some embodiments, the post-biotic compositions and/or personal care compositions thereof is formulated to inhibit the growth of C. striatum and increase the growth of S. epidermidis, such that the ratio of S. epidermidis to C. striatum is about 2 or more, based on a log count of C. striatum divided by a log count of S. epidermidis after incubating a 2 ml of a bacterial culture having a 0.1 optical density at 610 nm using a UV-VIS Spectrometer for C. striatum and S. epidermidis with a 2 ml of 1% of the post-biotic composition or personal care composition thereof at a temperature of 37° C. for 3 hours. For instance, the post-biotic compositions and/or personal care compositions thereof may inhibit the growth of C. striatum and increase the growth of S. epidermidis, such that the ratio of S. epidermidis to C. striatum is about 1.5 or more, about 1.75 or more, about 2 or more, about 2.3 or more, about 2.6 or more, about 2.9 or more, about 3.1 or more, about 3.4 or more, or any range thereof.

Additionally or alternatively, the post-biotic compositions and/or personal care compositions thereof may be formulated to inhibit the growth of E. coli and increase the growth of S. epidermidis, such that the ratio of S. epidermidis to E. coli is about 2 or more, based on a log count of E. coli divided by a log count of S. epidermidis after incubating a 2 ml of a bacterial culture having a 0.1 optical density at 610 nm using a UV-VIS Spectrometer for E. coli and S. epidermidis with a 2 ml of 1% of the post-biotic composition or personal care composition thereof at a temperature of 37° C. for 3 hours. For instance, the post-biotic compositions and/or personal care compositions thereof may inhibit the growth of E. coli and increase the growth of S. epidermidis, such that the ratio of S. epidermidis to E. coli is about 1.5 or more, about 1.75 or more, about 2 or more, about 2.3 or more, about 2.6 or more, about 2.9 or more, about 3.1 or more, about 3.4 or more, or any range thereof.

The post-biotic compositions and/or personal care compositions thereof may be formulated to inhibit the growth of S. aureus and increase the growth of S. epidermidis, such that the ratio of S. epidermidis to S. aureus is about 2 or more, based on a log count of S. aureus divided by a log count of S. epidermidis after incubating a 2 ml of a bacterial culture having a 0.1 optical density at 610 nm using a UV-VIS Spectrometer for S. aureus and S. epidermidis with a 2 ml of 1% of the post-biotic composition or personal care composition thereof at a temperature of 37° C. for 3 hours. For instance, the post-biotic compositions and/or personal care compositions thereof may inhibit the growth of S. aureus and increase the growth of S. epidermidis, such that the ratio of S. epidermidis to S. aureus is about 1.5 or more, about 1.75 or more, about 2 or more, about 2.3 or more, about 2.6 or more, about 2.9 or more, about 3.1 or more, about 3.4 or more, or any range thereof.

The personal care compositions may be in the form of skin care, hair care, sun care, or nail care compositions. Non-limiting examples of the form of the personal care compositions according to aspects of the invention include a shampoo, a hair conditioner, a lotion, a cream, a serum, a balm, a spray, a deodorant, an antiperspirant, a body wash, a shower gel, a bar soap, a soft soap, a sunscreen, a cosmetic, and the like. The personal care compositions may be in the form of an emulsion, such as an oil-in-water or water-in-oil emulsion.

Suitable components, such as those listed below, may be included or excluded from the formulations for the personal care compositions depending on the specific combination of other ingredients, the form of the personal care composition, and/or the use of the composition (e.g., a lotion, cream, spray, body wash, serum, etc.). For example, the personal care composition may include a hydrophilic phase and a hydrophobic phase when the personal care composition is in the form of an emulsion or a two-phase composition. In some embodiments, the personal care composition is in the form of a body wash, body lotion, or serum.

The post-biotic composition typically comprises one or more short chain fatty acid(s) and a plurality of amino acids. The inventors recognized that certain short chain fatty acid(s) and blends thereof can upregulate hydration related gene and tight junction gene and downregulated inflammation related gene expressions. For instance, it was determined that adding 0.6 wt. % of certain combinations of short chain fatty acid(s) into a commercially available body wash enabled the body wash to upregulate hydration related gene and tight junction gene and downregulated inflammation related gene expressions.

The short chain fatty acid(s) may comprise 6 or less carbon atoms. For example, short chain acids mean not only hydrocarbyl acids of formula CₙH₂ₙ+1COOH, wherein n is 0 to 6, but also short chained acids that are substituted with oxygen containing groups such as alcohols or oxo groups. The short chain fatty acid(s) disclosed herein may in some instances be in the form of a salt. The short chain fatty acid(s) may be selected from pentanoic acid, CH₃CH₂CH₂CH₂COOH, isovaleric acid, 3-methylbutanoic acid, (CH₃)₂CHCH₂COOH, 2-methylpropanoic acid, (CH₃)₂CHCOOH, butanoic acid, CH₃CH₂CH₂COOH, CH₃CH₂COOH, ethanoic acid, CH₃COOH, methanoic acid, HCOOH, acetic acid, butanedioic acid, succinic acid, formic acid, propionic acid, butyric acid, isobutyric acid, valeric acid, isovaleric acid, lactic acid, pyruvic acid, a derivative thereof, a salt thereof, and a combination of two or more thereof.

Preferably, the post-biotic composition comprises lactic acid, pyruvic acid, acetic acid, mandelic acid, a derivative thereof, a salt thereof, and a combination of two or more thereof. In some embodiments, the post-biotic composition may comprise one or more short chain fatty acid(s) selected from lactic acid, acetic acid, pyruvic acid, a derivative thereof, a salts thereof, and a combination of two or more thereof.

The inventors recognized that certain weight ratios of lactic acid and/or salts thereof to pyruvic acid and/or salts thereof may further enhance the growth of desirable bacteria and/or inhibit the growth of undesirable bacteria. For example, the post-biotic compositions may have lactic acid and/or salts thereof and pyruvic acid and/or salts in a weight ratio of about 6:1 to about 2:1. In some instances, the weight ratio of the weight ratio of lactic acid and/or salts thereof to pyruvic acid and/or salts thereof is from about 6:1 to about 2:1, about 5.75:1 to about 2.25:1, about 5.5:1 to about 2.5:1; about 5.25:1 to about 2.75:1, about 5:1 to about 3:1, about 4.75:1 to about 3.25:1, about 4.5:1 to about 3.5:1, or about 4.25:1 to about 3.75:1, including any ranges and subranges therebetween. In one embodiment, the weight ratio of lactic acid to pyruvic acid is about 4:1.

Additionally or alternatively, the post-biotic composition may be formulated to have certain weight ratios of lactic acid and/or salts thereof to acetic acid and/or salts thereof. For instance, the post-biotic compositions may have lactic acid and/or salts thereof and acetic acid and/or salts in a weight ratio of about 6:1 to about 2:1. In some instances, the weight ratio of the weight ratio of lactic acid and/or salts thereof to pyruvic acid and/or salts thereof is from about 6:1 to about 2:1, about 5.75:1 to about 2.25:1, about 5.5:1 to about 2.5:1; about 5.25:1 to about 2.75:1, about 5:1 to about 3:1, about 4.75:1 to about 3.25:1, about 4.5:1 to about 3.5:1, or about 4.25:1 to about 3.75:1, including any ranges and subranges therebetween. In one embodiment, the weight ratio of lactic acid to acetic acid is about 4:1.

The post-biotic composition, in some embodiments, may comprise one or more of the short chain fatty acid(s) discussed below.

Examples of short-chain acids comprising one carbon include methanoic acid, formic acid, and HCOOH. Examples of short-chain acids comprising two carbons include ethanoic acid, acetic acid, CH₃COOH, ethanedioic acid, oxalic acid, HOOCCOOH, oxoethanoic acid, glyoxylic acid, formylformic acid, OHCCOOH, 2-hydroxyethanoic acid, glycolic acid, dicarbonous acid, hydroxyacetic acid, and HOCH₂COOH.

Examples of short-chain acids comprising three carbons include propanoic acid, ethanecarboxylic acid, CH₃CH₂COOH, prop-2-enoic acid, acrylic acid, acroleic acid, ethylenecarboxylic acid, propene acid, vinylformic acid, CH₂═CH—COOH, 2-propynoic acid, propiolic acid, acetylene carboxylic acid, propargylic acid, CH≡C—COOH, propanedioic acid, malonic acid, methanedicarboxylic acid, HOOC—CH₂—COOH, 2-hydroxypropanedioic acid, tartronic acid, hydroxymalonic acid, HOOC—CHOH—COOH, oxopropanedioic acid, mesoxalic acid, ketomalonic acid, HOOC—CO—COOH, 2,2-dihydroxypropanedioic acid, dihydroxymalonic acid, mesoxalic acid monohydrate, HOOC—C(OH)₂—COOH, 2-oxopropanoic acid, pyruvic acid, α-ketopropionic acid, acetylformic acid, pyroracemic acid, CH₃—CO—COOH, 2-hydroxypropanoic acid, lactic acid, milk acid, CH₃—CHOH—COOH, 3-hydroxypropanoic acid, hydracrylic acid, CH₂OH—CH₂—COOH, 2,3-dihydroxypropanoic acid, glyceric acid, CH₂OH—CHOH—COOH, 2-oxiranecarboxylic acid, and glycidic acid.

Examples of short-chain acids comprising four carbons include butanoic acid, butyric acid, propanecarboxylic acid, CH₃(CH₂)₂COOH, 2-methylpropanoic acid, isobutyric acid, isobutanoic acid, (CH₃)₂CHCOOH, 2-oxobutanoic acid, alpha-ketobutyric acid, CH₃—CH₂—CO—COOH, 3-oxobutanoic acid, acetoacetic acid, CH₃CO—CH₂—COOH, 4-oxobutanoic acid, succinic semialdehyde, HC(O)—CH₂—CH₂—COOH, (E)-butenedioic acid, fumaric acid, trans-1,2-ethylenedicarboxylic acid, 2-butenedioic acid, trans-butenedioic acid, allomaleic acid, boletic acid, donitic acid, lichenic acid, HOOC—CH═CH—COOH, (Z)-butenedioic acid, maleic acid, cis-butenedioic acid, maleinic acid, toxilic acid, HOOC—CH═CH—COOH, oxobutanedioic acid, oxaloacetic acid, oxalacetic acid, oxosuccinic acid, HOOC—CH₂—CO—COOH, hydroxybutanedioic acid, malic acid, hydroxybutanedioic acid, HOOC—CH₂—CHOH—COOH, 2,3-dihydroxybutanedioic acid, tartaric acid, 2,3-dihydroxysuccinic acid, threaric acid, racemic acid, uvic acid, paratartaric acid, HOOC(CHOH)₂COOH, (E)-but-2-enoic acid, crotonic acid, trans-2-butenoic acid, beta-methylacrylic acid, 3-methylacrylic acid, (E)-2-butenoic acid, and CH₃—CH═CH—COOH.

Examples of short-chain acids comprising five carbons include pentanoic acid, valeric acid, valerianic acid, butane-1-carboxylic acid, CH₃(CH₂)₃COOH, 3-methylbutanoic acid, isovaleric acid, (CH₃)₂CH—CH₂—COOH, pentanedioic acid, glutaric acid, propane-1,3-dicarboxylic acid, 1,3-propanedicarboxylic acid, n-pyrotartaric acid, HOOC—(CH₂)₃—COOH, 2-oxopentanedioic acid, alpha-ketoglutaric acid, 2-ketoglutaric acid, α-ketoglutaric acid, 2-oxoglutaric acid, oxoglutaric acid, and HOOC—(CH₂)₂—CO—COOH.

The post-biotic composition may include the one or more short chain fatty acid(s) and/or salts thereof in an amount from about 50 to about 99 wt. %, based on the total weight of the post-biotic composition. In some instances, the total amount of short chain fatty acid(s) and/or salts thereof present in the post-biotic composition is from about 50 to about 96 wt. %, about 50 to about 93, about 50 to about 90 wt. % wt. %, about 50 to about 87 wt. %, about 50 to about 85 wt. %, about 50 to about 83 wt. %, about 50 to about 81 wt. %, about 50 to about 79 wt. %, about 50 to about 77 wt. %, about 50 to about 75 wt. %, about 50 to about 72 wt. %; from about 60 to about 99 wt. %, about 60 to about 96 wt. %, about 60 to about 93, about 60 to about 90 wt. % wt. %, about 60 to about 87 wt. %, about 60 to about 85 wt. %, about 60 to about 83 wt. %, about 60 to about 81 wt. %, about 60 to about 79 wt. %, about 60 to about 77 wt. %, about 60 to about 75 wt. %, about 60 to about 72 wt. %; from about 65 to about 99 wt. %, about 65 to about 96 wt. %, about 65 to about 93, about 65 to about 90 wt. % wt. %, about 65 to about 87 wt. %, about 65 to about 85 wt. %, about 65 to about 83 wt. %, about 65 to about 81 wt. %, about 65 to about 79 wt. %, about 65 to about 77 wt. %, about 65 to about 75 wt. %; from about 70 to about 99 wt. %, about 70 to about 96 wt. %, about 70 to about 93, about 70 to about 90 wt. % wt. %, about 70 to about 87 wt. %, about 70 to about 85 wt. %, about 70 to about 83 wt. %, about 70 to about 81 wt. %, about 70 to about 79 wt. %, about 70 to about 77 wt. %; from about 74 to about 99 wt. %, about 74 to about 96 wt. %, about 74 to about 93, about 74 to about 90 wt. % wt. %, about 74 to about 87 wt. %, about 74 to about 85 wt. %, about 74 to about 83 wt. %, about 74 to about 81 wt. %, about 74 to about 79 wt. %; from about 78 to about 99 wt. %, about 78 to about 96 wt. %, about 78 to about 93, about 78 to about 90 wt. % wt. %, about 78 to about 87 wt. %, about 78 to about 85 wt. %, about 78 to about 83 wt. %, about 78 to about 81 wt. %; from about 82 to about 99 wt. %, about 82 to about 96 wt. %, about 82 to about 93, about 82 to about 90 wt. % wt. %, about 82 to about 87 wt. %; from about 85 to about 99 wt. %, about 85 to about 96 wt. %, about 85 to about 93, about 85 to about 90 wt. % wt. %, or any range or subrange thereof, based on the total weight of the post-biotic composition.

In some embodiments, the post-biotic composition comprises two or more short chain fatty acids and/or salts thereof. For example, the post-biotic composition may include two, three, four, five, six, seven, eight, or any range or subrange thereof of short chain fatty acids and/or salts thereof. In at least one embodiment, the post-biotic composition comprises a first short chain fatty acid and/or salts thereof in an amount from about 50 to about 90 wt. % and a second short chain fatty acid and/or salts thereof in an amount from about 1 to about 50 wt. %, where the first and second short chain fatty acids and/or salts thereof are selected from those disclosed herein and the weight percentages are based on the total weight of the post-biotic composition. For example, the first short chain fatty acid and/or salts thereof may be present in the post-biotic composition in an amount from about from about 50 to about 87 wt. %, about 50 to about 85 wt. %, about 50 to about 83 wt. %, about 50 to about 81 wt. %, about 50 to about 79 wt. %, about 50 to about 77 wt. %, about 50 to about 75 wt. %, about 50 to about 72 wt. %; from about 60 to about 90 wt. % wt. %, about 60 to about 87 wt. %, about 60 to about 85 wt. %, about 60 to about 83 wt. %, about 60 to about 81 wt. %, about 60 to about 79 wt. %, about 60 to about 77 wt. %, about 60 to about 75 wt. %, about 60 to about 72 wt. %; from about 65 to about 90 wt. % wt. %, about 65 to about 87 wt. %, about 65 to about 85 wt. %, about 65 to about 83 wt. %, about 65 to about 81 wt. %, about 65 to about 79 wt. %, about 65 to about 77 wt. %, about 65 to about 75 wt. %; from about 70 to about 90 wt. % wt. %, about 70 to about 87 wt. %, about 70 to about 85 wt. %, about 70 to about 83 wt. %, about 70 to about 81 wt. %, about 70 to about 79 wt. %, about 70 to about 77 wt. %; from about 74 to about 90 wt. % wt. %, about 74 to about 87 wt. %, about 74 to about 85 wt. %, about 74 to about 83 wt. %, about 74 to about 81 wt. %, about 74 to about 79 wt. %; from about 78 to about 90 wt. % wt. %, about 78 to about 87 wt. %, about 78 to about 85 wt. %, about 78 to about 83 wt. %, about 78 to about 81 wt. %; from about 82 to about 90 wt. % wt. %, about 82 to about 87 wt. %; from about 85 to about 90 wt. % wt. %, or any range or subrange thereof, based on the total weight of the post-biotic composition.

The second short chain fatty acid and/or salts thereof may be present in the post-biotic composition in an amount from about 1 to about 50 wt. %, based on the total weight of the post-biotic composition. For instance, the second short chain fatty acid and/or salts thereof may be present in the post-biotic composition in an amount from about 1 to about 50 wt. %, about 1 to about 40 wt. %, about 1 to about 35 wt. %, about 1 to about 30 wt. %, about 1 to about 25 wt. %, about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %; from about 5 to about 50 wt. %, about 5 to about 40 wt. %, about 5 to about 35 wt. %, about 5 to about 30 wt. %, about 5 to about 25 wt. %, about 5 to about 20 wt. %, about 5 to about 18 wt. %, about 5 to about 16 wt. %, about 5 to about 14 wt. %, about 5 to about 12 wt. %, about 5 to about 10 wt. %; from about 9 to about 50 wt. %, about 9 to about 40 wt. %, about 9 to about 35 wt. %, about 9 to about 30 wt. %, about 9 to about 25 wt. %, about 9 to about 20 wt. %, about 9 to about 18 wt. %, about 9 to about 16 wt. %, about 9 to about 14 wt. %; from about 13 to about 50 wt. %, about 13 to about 40 wt. %, about 13 to about 35 wt. %, about 13 to about 30 wt. %, about 13 to about 25 wt. %, about 13 to about 20 wt. %, about 13 to about 18 wt. %, about 13 to about 16 wt. %; from about 15 to about 50 wt. %, about 15 to about 40 wt. %, about 15 to about 35 wt. %, about 15 to about 30 wt. %, about 15 to about 25 wt. %, about 15 to about 20 wt. %, about 15 to about 18 wt. %; from about 17 to about 50 wt. %, about 17 to about 40 wt. %, about 17 to about 35 wt. %, about 17 to about 30 wt. %, about 17 to about 25 wt. %; from about 19 to about 50 wt. %, about 19 to about 40 wt. %, about 19 to about 35 wt. %, about 19 to about 30 wt. %, about 19 to about 25 wt. %; from about 21 to about 50 wt. %, about 21 to about 40 wt. %, about 21 to about 35 wt. %, about 21 to about 30 wt. %, about 21 to about 25 wt. %; from about 25 to about 50 wt. %, about 25 to about 40 wt. %, about 25 to about 35 wt. %, about 25 to about 30 wt. %; from about 30 to about 50 wt. %, about 30 to about 40 wt. %, about 30 to about 35 wt. %; from about 35 to about 50 wt. %, about 35 to about 40 wt. %; from about 40 to about 50 wt. %, about 45 to about 50 wt. %, or any range or subrange thereof, based on the total weight of the post-biotic composition.

The post-biotic composition may be formulated to have certain weight ratios of the first short chain fatty acid and/or salts thereof to the second short chain fatty acid and/or salts thereof. For example, the post-biotic composition may have a weight ratio of the first short chain fatty acid and/or salts thereof to the second short chain fatty acid and/or salts thereof of from about 9:1 to about 2:1. In some embodiments, the post-biotic composition has a weight ratio of a first short chain fatty acid and/or salts thereof to a second short chain fatty acid and/or salts thereof that is from about 9:1 to about 2:1, about 8:1 to about 2:1, about 7:1 to about 2:1, about 6:1 to about 2:1, about 5:1 to about 2:1, about 4:1 to about 2:1; from about 9:1 to about 3:1, about 8:1 to about 3:1, about 7:1 to about 3:1, about 6:1 to about 3:1, about 5:1 to about 3:1, about 4:1 to about 3:1; from about 9:1 to about 4:1, about 8:1 to about 4:1, about 7:1 to about 4:1, about 6:1 to about 4:1, about 5:1 to about 4:1; from about 9:1 to about 5:1, about 8:1 to about 5:1, about 7:1 to about 5:1, about 6:1 to about 5:1; from about 9:1 to about 6:1, about 8:1 to about 6:1, about 7:1 to about 6:1, or any range and subrange thereof.

The post-biotic composition typically comprises a plurality of amino acids. The plurality of amino acids may comprise at least 2 amino acids selected from histidine, glycine, threonine, arginine, alanine, valine, proline, a salt thereof, and a combination of two or more thereof. For example, the plurality of amino acids may comprise at least 3 amino acids, at least 4 amino acids, at least 5 amino acids, or at least 6 amino acids selected from histidine, glycine, threonine, arginine, alanine, valine, proline, a salt thereof, and a combination of two or more thereof. In at least one embodiment, the plurality of amino acids consists essentially of or consists of at least 3 amino acids, at least 4 amino acids, at least 5 amino acids, or at least 6 amino acids selected from histidine, glycine, threonine, arginine, alanine, valine, proline, a salt thereof, and a combination of two or more thereof.

Preferably, the plurality of amino acids comprises one or more of glycine, arginine, alanine, or a combination of two or more thereof. In some embodiments, the plurality of amino acids comprises two or more of glycine, arginine, alanine, or a combination thereof. The post-biotic composition may include glycine, arginine, and alanine in some instances. In some embodiments, at least 50 wt. % of the plurality of amino acids is comprised of glycine, arginine, alanine, or a combination thereof, based on the total weight of the plurality of amino acids. For example, glycine, arginine, alanine, or a combination thereof may comprise at least 60 wt. %, at least 65 wt. %, at least 68 wt. %, at least 70 wt. %, at least 72 wt. %, at least 74 wt. %, at least 76 wt. %, at least 78 wt. %, at least 80 wt. %, at least 82 wt. %, at least 84 wt. %, at least 86 wt. %, at least 88 wt. %, or at least 90 wt. % of the plurality of amino acids, based on the total weight of the plurality of amino acids.

The plurality of amino acids may comprise one or more of glycine, arginine, alanine, or a combination of two or more thereof and optionally one or more of histidine, threonine, valine, proline, or a combination of two or more thereof. For example, the plurality of amino acids may comprise two or more amino acids selected from glycine, arginine, and alanine, and optionally two or more amino acids selected from histidine, threonine, valine, and proline.

The amount of amino acid in the post-biotic composition and/or a personal care composition thereof may be about 1 to about 25 wt. %, based on the total weight of the post-biotic composition. For example, in some instances, the amount of amino acid(s) present in the post-biotic composition is about 2 to about 25 wt. %, about 3 to about 25 wt. %, about 4 to about 25 wt. %, about 5 to about 25 wt. %, about 7.5 to about 25 wt. %, about 10 to about 25 wt. %, about 15 to about 25 wt. %, about 20 to about 25 wt. %; about 1 to about 20 wt. %, about 2 to about 20 wt. %, about 3 to about 20 wt. %, about 4 to about 20 wt. %, about 5 to about 20 wt. %, about 7.5 to about 20 wt. %, about 10 to about 20 wt. %, about 15 to about 20 wt. %; about 1 to about 15 wt. %, about 2 to about 15 wt. %, about 3 to about 15 wt. %, about 4 to about 15 wt. %, about 5 to about 15 wt. %, about 7.5 to about 15 wt. %, about 10 to about 15 wt. %; about 1 to about 10 wt. %, about 2 to about 10 wt. %, about 3 to about 10 wt. %, about 4 to about 10 wt. %, about 5 to about 10 wt. %, about 7.5 to about 10 wt. %; about 1 to about 7.5 wt. %, about 2 to about 7.5 wt. %, about 3 to about 7.5 wt. %, about 4 to about 7.5 wt. %, about 5 to about 7.5 wt. %; about 1 to about 5 wt. %, about 2 to about 5 wt. %, about 3 to about 5 wt. %, about 4 to about 5 wt. %; about 1 to about 4 wt. %, about 2 to about 4 wt. %, about 3 to about 4 wt. %, including all ranges and subranges thereof, based on the total weight of the post-biotic composition.

In accordance with another aspect of the invention, provided is a personal care composition. The personal care compositions disclosed herein typically includes a post-biotic system as disclosed herein, preferably in an effective amount. As noted above, the post-biotic composition is generally referred to as a post-biotic system when such post-biotic composition is incorporated as a component of a personal care composition.

The post-biotic system may be present in the personal care composition in an amount from about 0.01 to about 70 wt. %, based on the total weight of the personal care composition, depending on, e.g., the form of the personal care compositions and/or the other components and/or ingredients of the personal care composition. For instance, the personal care composition may include a post-biotic system in an amount from about 0.01 to about 10 wt. %, about 0.01 to about 9 wt. %, about 0.01 to about 8 wt. %, about 0.01 to about 7 wt. %, about 0.01 to about 6 wt. %, about 0.01 to about 5 wt. %, about 0.01 to about 4 wt. %, about 0.01 to about 3 wt. %, about 0.01 to about 2 wt. %, about 0.01 to about 1 wt. %, about 0.01 to about 0.8 wt. %, about 0.01 to about 0.6 wt. %, about 0.01 to about 0.5 wt. %, about 0.01 to about 0.4 wt. %, about 0.01 to about 0.3 wt. %; from about 0.05 to about 10 wt. %, about 0.05 to about 9 wt. %, about 0.05 to about 8 wt. %, about 0.05 to about 7 wt. %, about 0.05 to about 6 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.8 wt. %, about 0.05 to about 0.6 wt. %, about 0.05 to about 0.5 wt. %, about 0.05 to about 0.4 wt. %, about 0.05 to about 0.3 wt. %; from about 0.1 to about 10 wt. %, about 0.1 to about 9 wt. %, about 0.1 to about 8 wt. %, about 0.1 to about 7 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.8 wt. %, about 0.1 to about 0.6 wt. %, about 0.1 to about 0.5 wt. %, about 0.1 to about 0.4 wt. %, about 0.1 to about 0.3 wt. %; from about 0.2 to about 10 wt. %, about 0.2 to about 9 wt. %, about 0.2 to about 8 wt. %, about 0.2 to about 7 wt. %, about 0.2 to about 6 wt. %, about 0.2 to about 5 wt. %, about 0.2 to about 4 wt. %, about 0.2 to about 3 wt. %, about 0.2 to about 2 wt. %, about 0.2 to about 1 wt. %, about 0.2 to about 0.8 wt. %, about 0.2 to about 0.6 wt. %, about 0.2 to about 0.5 wt. %, about 0.2 to about 0.4 wt. %; from about 0.5 to about 10 wt. %, about 0.5 to about 9 wt. %, about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.8 wt. %; from about 0.75 to about 10 wt. %, about 0.75 to about 9 wt. %, about 0.75 to about 8 wt. %, about 0.75 to about 7 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; from about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 3 to about 10 wt. %, about 3 to about 9 wt. %, about 3 to about 8 wt. %, about 3 to about 7 wt. %, about 3 to about 6 wt. %, about 3 to about 5 wt. %; from about 5 to about 10 wt. %, about 5 to about 9 wt. %, about 5 to about 8 wt. %, about 5 to about 7 wt. %; from about 7 to about 10 wt. %, about 7 to about 9 wt. %; from about 8 to about 10 wt. %, including any range or subrange thereof, based on the total weight of the personal care composition.

The personal care compositions may be in a form that includes a higher amount of the post-biotic system, such as from about 10 wt. % to about 70 wt. %, based on the total weight of the personal care composition. For example, the post-biotic system may be present in the personal care composition in an amount from about 10 to about 60 wt. %, about 10 to about 50 wt. %, about 10 to about 40 wt. %, about 10 to about 30 wt. %, about 10 to about 20 wt. %; from about 20 to about 70 wt. %, about 20 to about 60 wt. %, about 20 to about 50 wt. %, about 20 to about 40 wt. %, about 20 to about 30 wt. %; from about 25 to about 70 wt. %, about 25 to about 60 wt. %, about 25 to about 50 wt. %, about 25 to about 40 wt. %, about 25 to about 30 wt. %; from about 30 to about 70 wt. %, about 30 to about 60 wt. %, about 30 to about 50 wt. %, about 30 to about 40 wt. %; from about 35 to about 70 wt. %, about 35 to about 60 wt. %, about 35 to about 50 wt. %, about 35 to about 40 wt. %; from about 40 to about 70 wt. %, about 40 to about 60 wt. %, about 40 to about 50 wt. %; from about 45 to about 70 wt. %, about 45 to about 60 wt. %, about 45 to about 50 wt. %; from about 50 to about 70 wt. %, about 50 to about 60 wt. %; from about 55 to about 70 wt. %, about 55 to about 60 wt. %; from about 60 to about 70 wt. %, about 65 to about 70 wt. %, including ranges or subranges thereof, based on the total weight of the personal care composition.

The personal care composition may include one or more polysaccharide(s). In some instances, the personal care compositions include polysaccharide(s) in an amount of about 0.1 to about 4 wt. %, about 0.1 to about 3 wt. %, about 0.1 to about 2 wt. %, about 0.1 to about 1 wt. %; from about 0.5 to about 4 wt. %, about 0.5 to about 3 wt. %, about 0.5 to about 2 wt. %, about 0.5 to about 1 wt. %; from about 1 to about 4 wt. %, about 1 to about 3 wt. %, about 1 to about 2 wt. %, or any range or subrange thereof, based on the total weight of the personal care composition.

In some embodiments, however, the personal care compositions may be formulated to have a limited and/or minimal amount of polysaccharides. For example, the total amount of polysaccharide(s) in the personal care composition may be about 5 wt. % or less, about 4 wt. % or less, about 3 wt. % or less, about 2 wt. % or less, about 1 wt. % or less, about 0.5 wt. % or less, about 0.1 wt. % or less, or about 0.05 wt. % or less, based on the total weight of the personal care composition. In some embodiments, the personal care compositions may be free or substantially free of polysaccharides.

Examples of polysaccharide(s) include, but are not limited to, saccharide isomerate, cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, carrageenan, tragacanth gum, xanthan gum, biosaccharide gum, inulin, and combinations of two or more thereof. The polysaccharide may be selected from fructan, inulin, carrageenan, and a combination of two or more thereof. Additionally or alternatively, the polysaccharide may comprise saccharide isomerate.

Additionally or alternatively, the personal care compositions may include a fatty alcohol. The amount of fatty alcohol in the personal care composition may vary, but in some instances is from about 0.01 to about 12 wt. %, based on the total weight of the personal care compositions. For example, the amount of fatty alcohol(s) present in the personal care composition may be from about 0.01 to about 12 wt. %, about 0.01 to about 10 wt. %, about 0.01 to about 8 wt. %, about 0.01 to about 6 wt. %, about 0.01 to about 5 wt. %, about 0.01 to about 4 wt. %, about 0.01 to about 3 wt. %, about 0.01 to about 2 wt. %, about 0.01 to about 1 wt. %; about 0.1 to about 12 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; about 0.5 to about 12 wt. %, about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 1 to about 12 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 12 wt. %, about 2 to about 10 wt. %, about 2 to about 8 wt. %, about 2 to about 6 wt. %, about 2 to about 5 wt. %, about 2 to about 4 wt. %; about 4 to about 12 wt. %, about 4 to about 10 wt. %, about 4 to about 8 wt. %, about 4 to about 6 wt. %, including ranges and subranges therebetween, based on the total weight of the personal care compositions.

The fatty alcohol(s) may be selected from decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a combination of two or more thereof. In at least one embodiment, the fatty alcohol is 2-butyloctanol.

Examples of additional fatty alcohols that are worth noting include certain saturated or an unsaturated fatty alcohols. The saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

Exemplary unsaturated liquid fatty alcohols generally include in their structure at least one double or triple bond. For example, the unsaturated fatty alcohol may include several double bonds (such as 2 or 3 double bonds), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol, and undecylenyl alcohol.

The fatty alcohols may be alkoxylated fatty alcohols, e.g., having about 1 to about 100 moles of an alkylene oxide per mole of alkoxylated fatty alcohol. As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are worth mentioning. In at least one instance, the one or more alkoxylated fatty alcohols include steareth-20. In some instances, the one or more alkoxylated fatty alcohols may be exclusively steareth-20.

The personal care composition may comprise fatty alcohol derivatives, such as methyl stearyl ether; 2-ethylhexyl dodecyl ether; stearyl acetate; cetyl propionate; the ceteth series of compounds, such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of branched alcohols, such as octyldodecyl alcohol, dodecylpentadecyl alcohol, hexyldecyl alcohol, and isostearyl alcohol; polyoxyethylene ethers of behenyl alcohol; PPG ethers, such as PPG-9-steareth-3, PPG-11 stearyl ether, PPG8-ceteth-1, and PPG-10 cetyl ether; and a combination of two or more thereof.

The personal care composition may include one or more polyol(s) in an amount that may vary depending on the form of the personal care composition and/or other components therein. The amount of polyols in the personal care composition may be from about 1 to about 30%, based on the total weight of the personal care composition. For example, the amount of polyol present in the personal care compositions may be from about 1 to about 30 wt. %, about 1 to about 25 wt. %, about 1 to about 20 wt. %, about 1 to about 15 wt. %, about 1 to about 10 wt. %, about 1 to about 8 wt. %, about 1 to about 6 wt. %, about 1 to about 4 wt. %; from about 5 to about 30 wt. %, about 5 to about 25 wt. %, about 5 to about 22 wt. %, about 5 to about 20 wt. %, about 5 to about 18 wt. %, about 5 to about 16 wt. %; from about 8 to about 30 wt. %, about 8 to about 25 wt. %, about 8 to about 22 wt. %, about 8 to about 20 wt. %, about 8 to about 18 wt. %, about 8 to about 16 wt. %; from about 10 to about 30 wt. %, about 10 to about 25 wt. %, about 10 to about 22 wt. %, about 10 to about 20 wt. %, about 10 to about 18 wt. %, about 10 to about 16 wt. %; from about 12 to about 30 wt. %, about 12 to about 25 wt. %, about 12 to about 22 wt. %, about 12 to about 20 wt. %, about 12 to about 18 wt. %, about 12 to about 16 wt. %; from about 14 to about 30 wt. %, about 14 to about 25 wt. %, about 14 to about 22 wt. %, about 14 to about 20 wt. %, about 14 to about 18 wt. %, about 14 to about 16 wt. %, including ranges and subranges thereof, based on the total weight of the personal care composition.

The polyol(s) may be chosen from glycols or compounds with numerous hydroxyl groups. The term “polyol” should be understood as meaning, within the meaning of the present disclosure, an organic molecule comprising at least two free hydroxyl groups. The polyols of the personal care composition may be glycols or compounds with numerous hydroxyl groups. In some cases, the one or more polyols is/are selected from the group consisting of C₂-C₃₂ polyols. The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, and dipropylene glycol, and mixtures thereof. In some cases, the polyol is propylene glycol. In some further cases, the personal care composition comprises at least propylene glycol, and optionally one or more polyols other than propylene glycol.

Non-limiting examples of polyols that may, optionally, be included in the personal care composition include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a combination of two or more thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or are chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and a mixture thereof. Non-limiting examples of polyols that may be included in the personal care composition, in certain instances, include ethylene glycol, propylene glycol, butylene glycol, hexylene glycol, diglycerin, diethylene glycol, and dipropylene glycol, and a combination of two or more thereof.

The personal care composition may include one or more polyol(s) that are humectants. Examples of humectants that may be incorporated in certain embodiments include triethylene glycol, tripropylene glycol, propylene glycol, glycerin, polyethylene glycols, polypropylene glycol, glycerin, sorbitol, hexylene glycol, butylene glycol, hyaluronic acid, 1,2 hexanediol, saccharide isomerate and a combination of two or more thereof. In some cases, the one or more humectants comprise at least one of glycerin, propylene glycol, or polyethylene glycol and, optionally, one or more additional humectants.

The personal care composition may include one or more fatty compound(s). In some instances, the personal care composition includes one or more fatty compounds in an amount from about 0.1 to about 20 wt. %, based on the total weight of the personal care composition. In some instances, the total amount of fatty compounds is about 0.1 to 20 wt. %, about 0.1 to about 18 wt. %, about 0.1 to about 16 wt. %, about 0.1 to about 14 wt. %, about 0.1 to about 12 wt. %, about 0.1 to about 10 wt. %, about 0.1 to about 8 wt. %, about 0.1 to about 7 wt. %, about 0.1 to about 6 wt. %, about 0.1 to about 5 wt. %; about 0.5 to 20 wt. %, about 0.5 to about 18 wt. %, about 0.5 to about 16 wt. %, about 0.5 to about 14 wt. %, about 0.5 to about 12 wt. %, about 0.5 to about 10 wt. %, about 0.5 to about 8 wt. %, about 0.5 to about 7 wt. %, about 0.5 to about wt. %, about 0.5 to about 5 wt. %; about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 7 wt. %, about 1 to about 6 wt. %, about 1 to about 5 wt. %; about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 7 wt. %, about 2 to about 6 wt. %, about 2 to about 5 wt. %; about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 7 wt. %, about 3 to about 6 wt. %, about 3 to about 5 wt. %; about 4 to about 20 wt. %, about 4 to about 18 wt. %, about 4 to about 16 wt. %, about 4 to about 14 wt. %, about 4 to about 12 wt. %, about 4 to about 10 wt. %, about 4 to about 8 wt. % about 4 to about 7 wt. %, about 4 to about 6 wt. %, about 4 to about 5 wt. %; about 5 to about 20 wt. %, about 5 to about 18 wt. %, about 5 to about 16 wt. %, about 5 to about 14 wt. %, about 5 to about 12 wt. %, about 5 to about 10 wt. %, or about 5 to about 8 wt. %, about 5 to about 7 wt. %, or about 5 to about 6 wt. %, including all ranges and sub-ranges thereof, based on the total weight of the personal care composition.

The fatty compounds, if present, may be chosen from oils, waxes, fatty alcohols, fatty acids, fatty esters, fatty ethers, triglyceride compounds, and a mixture thereof. The fatty compounds may include or be chosen from one or more oil(s). The term “oil” means any fatty substance that is in liquid form at room temperature (24-25° C.) and at atmospheric pressure (760 mmHg). The oil may be a hydrocarbon-based oil, such as a saturated hydrocarbon oil, an unsaturated hydrocarbon oil, lipids, triglycerides, a natural oil, and/or a synthetic oil. The one or more oil(s) may be synthetic oils and/or plant based oils. For instance, the oil may be a synthetic oil, such as a mineral oil and/or an alkane (paraffins). Light (low viscosity) highly branched hydrocarbon oils may also be suitable in some instances. In some embodiments, the oil is chosen from plant based or vegetable based oils, such as coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture thereof.

Additional skin and/or hair compatible oils include a range of liquid hydrocarbons, for example, linear and branched oils such as liquid paraffin, squalene, squalane, mineral oil, low viscosity synthetic hydrocarbons, such as polyalphaolefins and polybutene. Other useful skin and/or compatible oils may be silicone based, for example, linear and cyclic polydimethyl siloxane, organo functional silicones (alkyl and alkyl aryl), and amino silicones.

The fatty compounds may, in some instances, include or be chosen from one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (Helianthus annuus), carnauba, candelilla, ouricury or japan wax or cork fiber or sugarcane waxes), or a mixture thereof. In some embodiments, the fatty compound is a fatty ester selected from include glycerol fatty esters, sorbitan fatty esters, ethoxylated fatty esters, synthetic esters, and a combination of two or more thereof.

Additionally or alternatively, the personal care composition may include one or more thickener(s). The amount of thickener(s) in the composition may be about 1 to about 25 wt. %, based on the total weight of the personal care composition. In some instances, the total amount of the one or more thickener(s) present in the personal care composition is about 2 to about 25 wt. %, about 3 to about 25 wt. %, about 4 to about 25 wt. %, about 5 to about 25 wt. %, about 7.5 to about 25 wt. %, about 10 to about 25 wt. %, about 15 to about 25 wt. %, about 20 to about 25 wt. %; about 1 to about 20 wt. %, about 2 to about 20 wt. %, about 3 to about 20 wt. %, about 4 to about 20 wt. %, about 5 to about 20 wt. %, about 7.5 to about 20 wt. %, about 10 to about 20 wt. %, about 15 to about 20 wt. %; about 1 to about 15 wt. %, about 2 to about 15 wt. %, about 3 to about 15 wt. %, about 4 to about 15 wt. %, about 5 to about 15 wt. %, about 7.5 to about 15 wt. %, about 10 to about 15 wt. %; about 1 to about 10 wt. %, about 2 to about 10 wt. %, about 3 to about 10 wt. %, about 4 to about 10 wt. %, about 5 to about 10 wt. %, about 7.5 to about 10 wt. %; about 1 to about 7.5 wt. %, about 2 to about 7.5 wt. %, about 3 to about 7.5 wt. %, about 4 to about 7.5 wt. %, about 5 to about 7.5 wt. %; about 1 to about 5 wt. %, about 2 to about 5 wt. %, about 3 to about 5 wt. %, about 4 to about 5 wt. %; about 1 to about 4 wt. %, about 2 to about 4 wt. %, about 3 to about 4 wt. %, including all ranges and subranges therebetween, based on the total weight of the personal care composition.

The thickener(s) may be chosen from carboxylic acid polymers; crosslinked polyacrylate polymers; polyacrylamide polymers; gums, and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. Non-limiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and a combination of two or more thereof.

In certain cases, the personal care composition may be formulated to have thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (e.g., carboxymethyl cellulose, methyl cellulose, methylhydroxypropyl cellulose, hydroxyethyl cellulose, hydroxypropyl cellulose, cellulose sulfate sodium salt), and/or starch derivatives (e.g., starch oxide, dialdehyde starch, dextrin, British gum, acetyl starch, starch phosphate, carboxymethyl starch, hydroxyethyl starch, hydroxypropyl starch). In some embodiments, the thickeners are selected from among hydroxypropyl methylcellulose, polyethylene glycols (PEGs), polyacrylic acids, cross-linked homopolymer of acrylic acid, and acrylates of C₁₀₋₃₀ alkyl acrylate crosspolymer.

The personal care composition may, in certain embodiments, comprise a surfactant. The surfactant can be any anionic, nonionic, amphoteric, or zwitterionic surfactant, or combinations thereof. The amount of surfactant(s) in the composition may be about 0.1 to about 25 wt. % or about 1 to about 25 wt. %, based on the total weight of the personal care composition. In some instances, the amount of surfactant(s) present in the personal care composition is about 2 to about 25 wt. %, about 3 to about 25 wt. %, about 4 to about 25 wt. %, about 5 to about 25 wt. %, about 7.5 to about 25 wt. %, about 10 to about 25 wt. %, about 15 to about 25 wt. %, about 20 to about 25 wt. %; about 1 to about 20 wt. %, about 2 to about 20 wt. %, about 3 to about 20 wt. %, about 4 to about 20 wt. %, about 5 to about 20 wt. %, about 7.5 to about 20 wt. %, about 10 to about 20 wt. %, about 15 to about 20 wt. %; about 1 to about 15 wt. %, about 2 to about 15 wt. %, about 3 to about 15 wt. %, about 4 to about 15 wt. %, about 5 to about 15 wt. %, about 7.5 to about 15 wt. %, about 10 to about 15 wt. %; about 1 to about 10 wt. %, about 2 to about 10 wt. %, about 3 to about 10 wt. %, about 4 to about 10 wt. %, about 5 to about 10 wt. %, about 7.5 to about 10 wt. %; about 1 to about 7.5 wt. %, about 2 to about 7.5 wt. %, about 3 to about 7.5 wt. %, about 4 to about 7.5 wt. %, about 5 to about 7.5 wt. %; about 1 to about 5 wt. %, about 2 to about 5 wt. %, about 3 to about 5 wt. %, about 4 to about 5 wt. %; about 1 to about 4 wt. %, about 2 to about 4 wt. %, about 3 to about 4 wt. %, including all ranges and subranges thereof, based on the total weight of the personal care composition.

In at least one embodiment, multiple surfactants are used to achieve desired product qualities. A primary surfactant provides good foaming ability and cleaning effectiveness, while a secondary surfactant adds qualities of mildness to prevent irritation or over-drying of the skin. To prevent ingredients from separating, emulsifiers such as diethanolamine may be added.

A variety of anionic surfactants can be utilized in the personal care composition including, for example, long chain alkyl (C₆-C₂₂) materials such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required. These surfactants are typically highly water soluble as their sodium, potassium, alkyl and ammonium or alkanol ammonium containing salt form and can provide high foaming cleansing power. Other equivalent anionic surfactants may be used. In one embodiment, the anionic surfactant comprises sodium laureth sulfate, sodium pareth sulfate, and combinations thereof. The anionic surfactants may be in a salt form.

In one embodiment, anionic surfactants are present in the composition in an amount of 0 to about 15% by weight. For example, the anionic surfactants may be present in the personal care composition in an amount from about 0.5 to about 15 wt. %, about 0.5 to about 12 wt. %, about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 15 wt. %, about 1 to about 12 wt. %, about 1 to about 10 wt. %, about 1 to about 8 wt. %, about 1 to about 6 wt. %, about to about 4 wt. %, about 1 to about 2 wt. %; from about 2 to about 15 wt. %, about 2 to about 12 wt. %, about 2 to about 10 wt. %, about 2 to about 8 wt. %, about 2 to about 6 wt. %, about 2 to about 4 wt. %; from about 4 to about 15 wt. %, about 4 to about 12 wt. %, about 4 to about 10 wt. %, about 4 to about 8 wt. %, about 4 to about 6 wt. %; from about 6 to about 15 wt. %, about 6 to about 12 wt. %, about 6 to about 10 wt. %, about 6 to about 8 wt. %; from about 8 to about 15 wt. %, about 8 to about 12 wt. %, about 8 to 10 wt. %, or any range or subrange thereof, based on the total weight of the personal care composition. In one embodiment, anionic surfactants are present in an amount of about 6 to about 8 wt. %, based on the total weight of the personal care composition.

Amphoteric surfactants may also be included in the composition. These surfactants are typically characterized by a combination of high surfactant activity, lather forming and mildness. Amphoteric surfactants include, but are not limited to derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate. Examples of such compounds include sodium 3-dodecyaminopropionate, sodium 3-dodecylaminopropane sulfonate, N-alkyl taurines and N-higher alkyl aspartic acids. Additional examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine, sulfobetaines such as coco dimethyl sulfopropyl betaine, stearyl dimethyl sulfopropyl betaine, amido betaines, amidosulfobetaines and the like. Betaines having a long chain alkyl group, particularly coco, may be particularly useful as are those that include an amido groups such as the cocamidopropyl and cocoamidoethyl betaines. The amphoteric surfactant(s) may be in the form of a salt.

The amphoteric surfactants may be present in the personal composition in an amount from 0 to about 15 wt. %, based on the total weight of the personal care composition. For example, the amphoteric surfactants may be present in the personal care composition in an amount from about 0.5 to about 15 wt. %, about 0.5 to about 12 wt. %, about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 15 wt. %, about 1 to about 12 wt. %, about 1 to about 10 wt. %, about 1 to about 8 wt. %, about 1 to about 6 wt. %, about to about 4 wt. %, about 1 to about 2 wt. %; from about 2 to about 15 wt. %, about 2 to about 12 wt. %, about 2 to about 10 wt. %, about 2 to about 8 wt. %, about 2 to about 6 wt. %, about 2 to about 4 wt. %; from about 4 to about 15 wt. %, about 4 to about 12 wt. %, about 4 to about 10 wt. %, about 4 to about 8 wt. %, about 4 to about 6 wt. %; from about 6 to about 15 wt. %, about 6 to about 12 wt. %, about 6 to about 10 wt. %, about 6 to about 8 wt. %; from about 8 to about 15 wt. %, about 8 to about 12 wt. %, about 8 to 10 wt. %, or any range or subrange thereof, based on the total weight of the personal care composition. In one embodiment, the amphoteric surfactants are present in the personal composition in an amount of about 4 to about 6 wt. %, based on the total weight of the personal care composition.

The personal care composition may include one or more nonionic surfactant(s), Examples of nonionic surfactants include polysorbate 20, long chain alkyl glucosides having C₈-C₂₂ alkyl groups; coconut fatty acid monoethanolamides such as cocamide MEA; coconut fatty acid diethanolamides, fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (for example the PLURONIC™ block copolymers commercially available from BASF); fatty acid alkylolamides, (fatty acid amide polyethylene glycols); N-alkyl-, N-alkoxypolyhydroxy fatty acid amides; sucrose esters; sorbitol esters; polyglycol ethers; and combinations thereof.

The nonionic surfactant(s) may be selected from secondary alcohol ethoxylates, polyoxyethylene stearyl ethers, polyoxyethylene ethers, and mixtures of two or more thereof. The personal care compositions may include a nonionic surfactant blend including two or more fatty alcohol ethoxylates, each having a polyethylene oxide chain length of at least 2. Preferred fatty alcohol ethoxylates generally have a fatty alcohol chain length of C₁₂ to C₂₄, a degree of unsaturation of 0-2, and a polyethylene oxide chain length of 2 to 150 ethylene oxide units. For example, the fatty alcohol ethoxylates may have the general formula: CH₃—(CH₂)ₓ—CH₂—O—(CH₂—CH₂—O—)_{y}H—, where X=10-20, Y=2-100. In some cases, the blend of fatty alcohol ethoxylates include at least one fatty alcohol ethoxylate having a long polyethylene oxide chain length and at least one fatty alcohol ethoxylate having a short polyethylene oxide chain length. Suitable long chain length fatty alcohol ethoxylates have a polyethylene oxide chain length greater than 20, preferably 21 to 150, more preferably 21 to 100.

Examples of long chain fatty alcohol ethoxylates include Steareth-100 (100 indicates the polyethylene oxide chain length) and Steareth 21. Other long chain fatty alcohol ethoxylates may be used, e.g., Ceteth-100, Oleth-100 Myreth-100, and Beheneth-100. Examples of short chain length fatty alcohol ethoxylates include those having a polyethylene oxide chain length of less than or equal to 20, preferably 2 to 20. For example, the short chain length fatty alcohol ethoxylates may be selected from Steareth-2, Steareth-10, Ceteth-20, Steareth-20, Myreth-20, Oleth-20 and Beheneth-20.

The nonionic surfactants may be selected from polyoxyethylene ethers, such as polyoxyethylene stearyl ethers compounds. The polyoxyethylene ethers may comprise a mixture of high molecular mass saturated fatty alcohols, such as cetyl alcohol and stearyl alcohol, and ethylene oxide. The polyoxyethylene ethers may be selected from Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, and a combination of two or more thereof. Examples of polyoxyethylene stearyl ethers compounds include Steareth-2, Steareth-4, Steareth-6, Steareth-7, Steareth-21, Steareth-10, Steareth-11, Steareth-13, Steareth-15, Steareth-20, or combinations of two or more thereof. The nonionic surfactant(s) may be in the form of a salt.

The nonionic surfactants may be present in the personal composition in an amount of 0 to about 15 wt. %, based on the total weight of the personal care composition. In one embodiment, nonionic surfactants are present in the personal composition in an amount from about 0.5 to about 15 wt. %, about 0.5 to about 12 wt. %, about 0.5 to about 10 wt. %, about 0.5 to about 8 wt. %, about 0.5 to about 6 wt. %, about 0.5 to about 4 wt. %, about 0.5 to about 2 wt. %, about 0.5 to about 1 wt. %; from about 1 to about 15 wt. %, about 1 to about 12 wt. %, about 1 to about 10 wt. %, about 1 to about 8 wt. %, about 1 to about 6 wt. %, about to about 4 wt. %, about 1 to about 2 wt. %; from about 2 to about 15 wt. %, about 2 to about 12 wt. %, about 2 to about 10 wt. %, about 2 to about 8 wt. %, about 2 to about 6 wt. %, about 2 to about 4 wt. %; from about 4 to about 15 wt. %, about 4 to about 12 wt. %, about 4 to about 10 wt. %, about 4 to about 8 wt. %, about 4 to about 6 wt. %; from about 6 to about 15 wt. %, about 6 to about 12 wt. %, about 6 to about 10 wt. %, about 6 to about 8 wt. %; from about 8 to about 15 wt. %, about 8 to about 12 wt. %, about 8 to 10 wt. %, or any range or subrange thereof, based on the total weight of the personal care composition.

Cationic surfactants can also be included in the personal care composition. The cationic surfactants, if present, may include or be chosen from quaternary ammonium compounds, amidoamines, or a mixture thereof. Examples of cationic surfactants that may be suitable for the personal care composition include or may be chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and a combination of two or more thereof.

Additional, non-limiting examples of cationic surfactants include: cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride. Further cationic surfactants that may be included or excluded in certain embodiments include quaterniums, such as quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, or a combination of two or more thereof. Other cationic surfactants worth mentioning include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and a combination of two or more thereof.

The cationic surfactants may be present in the composition in an amount of 0 to about 2% by weight. For example, the amount of cationic surfactants in the personal care composition may be from about 0.05 to about 2 wt. %, about 0.05 to about 1.8 wt. %, about 0.05 to about 1.6 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 2 wt. %, about 0.1 to about 1.8 wt. %, about 0.1 to about 1.6 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %; from about 0.3 to about 2 wt. %, about 0.3 to about 1.8 wt. %, about 0.3 to about 1.6 wt. %, about 0.3 to about 1.4 wt. %, about 0.3 to about 1.2 wt. %, about 0.3 to about 1 wt. %, about 0.3 to about 0.8 wt. %; from about 0.6 to about 2 wt. %, about 0.6 to about 1.8 wt. %, about 0.6 to about 1.6 wt. %, about 0.6 to about 1.4 wt. %, about 0.6 to about 1.2 wt. %, about 0.6 to about 1 wt. %; from about 0.9 to about 2 wt. %, about 0.9 to about 1.8 wt. %, about 0.9 to about 1.6 wt. %, about 0.9 to about 1.4 wt. %; from about 1.2 to about 2 wt. %, about 1.2 to about 1.8 wt. %, about 1.2 to about 1.6 wt. %; from about 1.5 to about 2 wt. %, about 1.5 to about 1.8 wt. %, or any range or subrange thereof, based on the total weight of the personal care composition. In one embodiment, cationic surfactants are present in the composition in an amount of about 0.1 to about 0.3% by weight.

The personal care compositions, in certain embodiments, may further comprise a cationic polymer. The cationic polymers can comprise mixtures of monomer units derived from amine- and/or quaternary ammonium-substituted monomer and/or compatible spacer monomers. Suitable cationic polymers include, for example: copolymers of 1-vinyl-2-pyrrolidine and 1-vinyl-3-methyl-imidazolium salt (e.g., chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic polymers that may be used include polysaccharide polymers, such as cationic cellulose derivatives and cationic starch derivatives. Cationic celluloses include salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

The personal care composition may include or be selected from polyquaterniums. In one instance, the one or more cationic polymer(s) is selected from polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, guar hydroxypropyltrimonium chloride, and a combination of two or more thereof.

The personal care composition may include alpha hydroxy acids, beta hydroxy acids, polyhydroxy acids or combinations thereof. For instance, the personal care composition may include one or more one or more alpha hydroxy acid(s), such as those selected from C₃ to C₇ alpha-hydroxy acid or C₄ to C₆ alpha-hydroxy acid. The alpha hydroxy acid may be in the form of a salt, such as a sodium salt or a potassium salt. In at least one embodiment, the salt is a sodium salt (i.e., the cation associated with the salt is a sodium). Non-limiting examples of alpha hydroxy acids include, but are not limited to, mandelic acid, glycolic acid, citric acid, lactic acid, malic acid, tartaric acid, phytic acid, hydroxycaprylic acid, hydroxycapric acid, glutaric acid, gluconic acid, or a combination of two or more thereof. In some embodiments, the alpha hydroxy acid(s) is selected from among mandelic acid, glycolic acid, citric acid, lactic acid, malic acid, tartaric acid, phytic acid, hydroxycaprylic acid, hydroxycapric acid and combinations thereof. In further embodiments, the alpha hydroxy acids are citric acid, mandelic acid, glycolic acid, lactic acid or a combination of two or more thereof. In at least one embodiment, the alpha hydroxy acid is lactic acid.

The amount of alpha hydroxy acid(s) and/or a salt(s) thereof present in the personal care composition may range from about 0.1 to about 6 wt. %, based on the total weight of the personal care composition. For instance, the personal care compositions may include one or more alpha hydroxy acid(s) and/or a salt(s) thereof in an amount from about 0.1 to about 5 wt. %, about 0.1 to about 4 wt. %, about 0.1 to about 3 wt. %, about 0.1 to about 2 wt. %; from about 0.5 to about 6 wt. %, about 0.5 to about 5 wt. %, about 0.5 to about 4 wt. %, about 0.5 to about 3 wt. %, about 0.5 to about 2 wt. %; from about 1 to about 6 wt. %, about 1 to about 5 wt. %, about 1 to about 4 wt. %, about 1 to about 3 wt. %, about 1 to about 2 wt. %; from about 2 to about 6 wt. %, about 2 to about 5 wt. %, about 2 to about 4 wt. %, about 2 to about 3 wt. %; from about 3 to about 6 wt. %, about 3 to about 5 wt. %, about 3 to about 4 wt. %, about 0.1 to about 3 wt. %; from about 4 to about 6 wt. %, about 4 to about 5 wt. %, or any range or subrange thereof, based on the total weight of the personal care composition.

The personal care compositions can comprise one or more beta hydroxy acid(s) or a salt thereof. Non-limiting examples of beta hydroxy acids include salicylic acid, propionic acid, beta-hydroybutyric acid, beta-hydroxy beata-methylbutyric acid, carnitine, and combinations of two or more thereof. The beta hydroxy acids may in some cases be selected from salicylic acid, esters of salicylic acid, sodium salicylate, beta hydroxybutanoic acid, tropic acid, trethocanic acid, beta hydroxy acids obtained from white willow bark extract and/or wintergreen leaves, and combinations of two or more thereof.

The amount of beta hydroxy acid(s) or salt(s) thereof in the personal care composition may be from about 0.05 to about 2 wt. %, about 0.05 to about 1.8 wt. %, about 0.05 to about 1.6 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 2 wt. %, about 0.1 to about 1.8 wt. %, about 0.1 to about 1.6 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %; from about 0.3 to about 2 wt. %, about 0.3 to about 1.8 wt. %, about 0.3 to about 1.6 wt. %, about 0.3 to about 1.4 wt. %, about 0.3 to about 1.2 wt. %, about 0.3 to about 1 wt. %, about 0.3 to about 0.8 wt. %; from about 0.6 to about 2 wt. %, about 0.6 to about 1.8 wt. %, about 0.6 to about 1.6 wt. %, about 0.6 to about 1.4 wt. %, about 0.6 to about 1.2 wt. %, about 0.6 to about 1 wt. %; from about 0.9 to about 2 wt. %, about 0.9 to about 1.8 wt. %, about 0.9 to about 1.6 wt. %, about 0.9 to about 1.4 wt. %; from about 1.2 to about 2 wt. %, about 1.2 to about 1.8 wt. %, about 1.2 to about 1.6 wt. %; from about 1.5 to about 2 wt. %, about 1.5 to about 1.8 wt. %, or any range or subrange thereof, based on the total weight of the personal care composition.

The personal care composition can comprise one or more polyhydroxy acid(s) or a salt thereof. The polyhydroxy acids may include, but are not limited to, gluconolactone, gluconic acid, galactose, lactobionic acid, or combinations thereof. In some embodiments, the polyhydroxy acids are gluconolactone, lactobionic acid or combinations thereof. For instance, the polyhydroxy acid may be glucanodeltalactone. The amount of polyhydroxy acid(s) and/or a salt(s) thereof present in the personal care composition may range from about 0.1 to about 6 wt. %, based on the total weight of the personal care composition. For instance, the personal care compositions may include one or more polyhydroxy acid(s) and/or a salt(s) thereof in an amount from about 0.1 to about 5 wt. %, about 0.1 to about 4 wt. %, about 0.1 to about 3 wt. %, about 0.1 to about 2 wt. %; from about 0.5 to about 6 wt. %, about 0.5 to about 5 wt. %, about 0.5 to about 4 wt. %, about 0.5 to about 3 wt. %, about 0.5 to about 2 wt. %; from about 1 to about 6 wt. %, about 1 to about 5 wt. %, about 1 to about 4 wt. %, about 1 to about 3 wt. %, about 1 to about 2 wt. %; from about 2 to about 6 wt. %, about 2 to about 5 wt. %, about 2 to about 4 wt. %, about 2 to about 3 wt. %; from about 3 to about 6 wt. %, about 3 to about 5 wt. %, about 3 to about 4 wt. %, about 0.1 to about 3 wt. %; from about 4 to about 6 wt. %, about 4 to about 5 wt. %, or any range or subrange thereof, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

In certain embodiments, the personal care composition has a pH of from about 3.5 to about 10.0. In other embodiments, the personal care composition has a pH of from about 3.5 to about 8.0. In some embodiments, the personal care composition has a pH of from about 3.5 to about 7.5. In other embodiments, the personal care composition has a pH of from about 3.5 to about 7.0. In further embodiments, the personal care composition has a pH of from about 3.5 to about 6.5. Still other embodiments provide personal care compositions having a pH of from about 3.5 to about 6.0. While other embodiments provide personal care compositions having a pH of from about 3.5 to about 5.5. Yet other embodiments provide personal care compositions having a pH of from about 3.5 to about 5.0. In certain embodiments, the personal care composition has a pH of from about 3.75 to about 4.5. In certain embodiments, the personal care composition is a body wash, having a pH of from about 4.0 to about 6.0. In other embodiments, the personal care composition is a body lotion, having a pH of from about 4.0 to about 5.0.

Additional ingredients may be present in the personal care composition. These additional ingredients include water, proteins, vitamins, emollients, and botanicals, as well as ingredients to thicken, preserve, emulsify, add fragrance, adjust the pH, and color. The personal care compositions may include any of the following additional ingredients in an amount of from about 0.01 to about 5 wt. %. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

The personal care composition can comprise additional ingredients, such as proteins (e.g., hydrolyzed vegetable protein, hydrolyzed wheat protein, hydrolyzed milk protein, hydrolyzed silk and hydrolyzed collagen), vitamins (e.g., panthenol, biotin, vitamin E acetate, vitamin A and D palmitate), moisturizers/humectants (e.g., glycerin, propylene glycol, sodium pyroglutamic acid (also known as PCA), amino acid-based surfactants, and HLA), emollients (e.g., esters, isopropyl myristate, decyl oleate, C12-15 alkyl benzoate), botanicals (e.g., chamomile, aloe, rosemary), as well as preservatives, dyes, pH adjusters and chelating agents. Additional examples of vitamins that may be included in some personal care compositions include tocopherol, retinol, and ascorbic acid. Vitamin derivatives, such as ascorbyl monopalmitate, tocopheryl acetate, and Vitamin E palmitate, may also be included in certain embodiments of the invention.

Non-limiting examples of fragrances and perfumes include odor compounds selected from: 7-acetyl-1,2,3,4,5,6,7,8-octahydro-1,1,6,7-tetramethylnaphthalene, α-ionone, (3-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl 0-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl)propionaldehyde, ethylvanillin, heliotropin, hexylcinnamaldehyde, amylcinnamaldehyde, 2-methyl-2-(isopropylphenyl)propionaldehyde, coumarin, 7-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The personal care composition may further comprise one or more colorants. The colorants may be a pigment, a dye, or mixtures thereof. Non-limiting examples of pigments include titanium dioxide, Zinc Oxide, Kaolin, Mica etc. Non-limiting examples of dyes include food dyes suitable for food, drug and cosmetic applications, and mixtures thereof. Some color agents (colorants) are known as FD&C dyes.

The colorants may be present in any of the amounts disclosed above with respect to the additional ingredient and/or in an amount ranging from about 0.0001 wt. % to about 0.4 wt. %, including all percentages and subranges therebetween, based on the total weight of the personal care composition. In some embodiments, the colorants may be present in an amount ranging from about 0.0001 wt. % to about 4 wt. %, including all percentages and subranges therebetween, based on the total weight of the personal care composition. In certain embodiments, in order to prevent ingredients from separating, emulsifiers may be added.

In other embodiments, the personal composition may include and additional ingredient selected from any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); sequestrants, for example, tetrasodium EDTA, and/or their equivalents (0 to 2% by weight); biocides, for example, Triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight); preservatives, for example, phenoxyethanol, formaldehyde solution, parabens, pentanediol or sorbic acid (0 to 2% by weight); pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight); and dyes and pigments that are approved and suitable for cosmetic purposes.

The personal care compositions may be aqueous and, e.g., include water in an amount from about 1 to about 95 wt. %, based on the total weight of the personal care composition. For example, the water may be present in the personal care composition in an amount from about 5 to about 95 wt. %, about 10 to about 95 wt. %, about 15 to about 95 wt. %, about 20 to about 95 wt. %, about 30 to about 95 wt. %, about 40 to about 95 wt. %, about 50 to about 95 wt. %, about 60 to about 95 wt. %, about 70 to about 95 wt. %, about 80 to about 95 wt. %, about 90 to about 95 wt. %; about 5 to about 90 wt. %, about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 5 to about 80 wt. %, about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 5 to about 70 wt. %, about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %; about 5 to about 60 wt. %, about 10 to about 60 wt. %, about 15 to about 60 wt. %, about 20 to about 60 wt. %, about 30 to about 60 wt. %, about 40 to about 60 wt. %, about 50 to about 60 wt. %; about 5 to about 50 wt. %, about 10 to about 50 wt. %, about 15 to about 50 wt. %, about 20 to about 50 wt. %, about 30 to about 50 wt. %, about 40 to about 50 wt. %, including any ranges and subranges thereof, based on the total weight of the personal care composition.

In some embodiments, the personal care compositions are substantially free of water or are free of water. For instance, the personal care compositions may include about 5 wt. % or less, about 4 wt. % or less, about 3 wt. % or less, about 2 wt. % or less, about 1 wt. % or less, or about 0.5 wt. % or less of water, based on the total weight of the personal care compositions. In one embodiment, the personal care composition has about 0 wt. % of water, based on the total weight of the personal care compositions.

Aspects of the invention are directed to methods for improving skin by applying one or more personal care compositions. In some embodiments, the methods include applying one or more (e.g., two, three, four, five, six, or seven personal care compositions). The methods may employ the personal care compositions disclosed herein.

The methods and personal care compositions disclosed herein may be used to alleviate, mitigate, and/or treat skin suffering from dryness, hypersensitivity, atopic dermatitis, acne, psoriasis, and the like.

### EXAMPLES

### Example 1

Non-limiting example, post-biotic compositions (Systems A-C) were prepared in accordance with the formulations shown in Table 1.

TABLE 1

| INCI | System A | System B | System C |
|---|---|---|---|
| Compounds | (wt. %) | (wt. %) | (wt. %) |
| Lactic acid | 70 | 80 | 64 |
| Acetic acid | 15 | | |
| Pyruvic acid | | | 16 |
| Histidine | 0.4 | 1 | 1 |
| Glycine | 5 | 6 | 6 |
| Threonine | 0.6 | 1 | 1 |
| Arginine | 3.5 | 4 | 4 |
| Alanine | 3.5 | 4.5 | 4.5 |
| Valine | 1 | 2 | 2 |
| Proline | 1 | 1.5 | 1.5 |

### Example 2

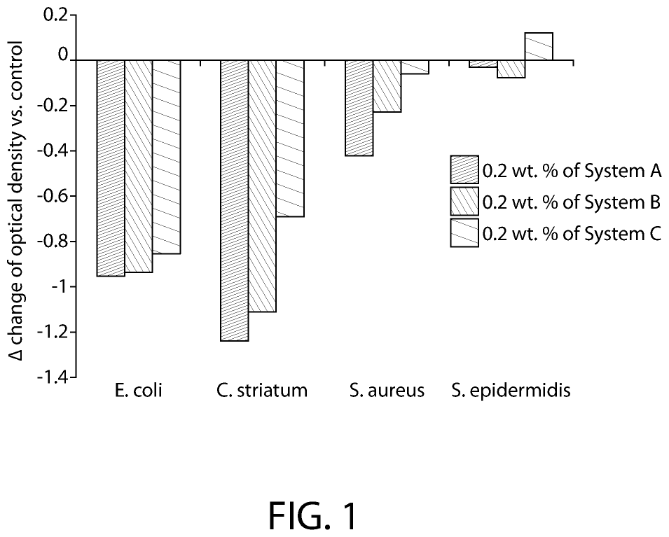

Growth inhibition assays were conducted to assess the effect of Systems A-C on skin bacteria. Specifically, a growth inhibition assay was conducted to test whether Systems A-C inhibited or promoted the growth of skin bacteria. A pure colony for each of E. coli, C. striatum, S. aureus, and S. epidermidis was grown in tryptic soy broth (“TSB”) medium overnight. The turbidity of the bacterial culture solution was adjusted to an optical density (“OD”) of 0.1 at 610 nm using a UV-VIS Spectrometer. Subsequently, 2 milliliter (“ml”) of the bacteria culture solution was incubated with 2 ml solution containing 0.2 wt. % of either System A, System B, or System C at a temperature of 37° C. for 48 hours. A sample of the bacteria incubated with medium alone was also prepared for a control. The turbidity of each tube was determined after incubation. Replicates were run for each treatment. If the value was negative, the system inhibited the growth of the respective growth and if the value was positive, the system promoted the growth of the respective bacteria. It was determined that at 48 hours of incubation, the solutions containing 0.2 wt. % of System A, System B, or System C inhibited the growth of E. coli, C. striatum, and S. aureus, but generally maintained or promoted S. epidermidis, as seen in FIG. 1.

### Example 3

Ex vivo skin tissue analysis was conducted to evaluate the effect on skin barrier property and anti-aging benefit of personal care compositions according to aspects of the invention. A solution containing 0.2 wt. % of System C with the remainder being water; a solution containing 0.5 wt. % of System B with the remainder being water; a solution containing 0.5 wt. % of System C with the remainder being water; and a solution containing retinoic acid were prepared. The solution containing 0.05 wt. % of retinoic acid with the remainder being water was evaluated as a positive control.

The skin samples were cut in pieces of approximately 8 mm by 3 mm (Ø×thickness) and cultured up to planned endpoints. 6 skin samples were used for each treatment. The skin samples were cultured in an air-liquid interface in a perforated ring of stainless steel in contact with culture medium (modified Williams' E medium) up to planned endpoints. Informed consent from the donor was obtained and retained by the clinic.

The solutions prepared in this Example were individually applied topically to respective skin samples. The application of the solutions was performed as follows: (1) skin samples were gently cleaned with a cotton pad, (2) a 4 μl sample of each solution was applied on top of the respective skin samples, and (3) the skin samples were then covered with a 6 Ø mm delivery membrane.

The level of filaggrin expression was evaluated in the skin samples. Specifically, twelve sections of the skin samples were immunostained with an antibody. The amount of the antigen present in each slide was evaluated by estimating the intensity and the distribution of the pink/red coloration within the epidermis using Image J (NIH-USA). The obtained data was normalized upon the dimension of the analyzed surface expressed in pixels.

The total level of collagen expression was also evaluated in the skin samples. In particular, skin sections were stained with Picrosirius Red histochemical staining, which dyes collagen fibers in purple-red. The papillary dermis was selected for the analysis, being the portion of the dermis that is more subjected to variation in response to application of personal care compositions.

A deconvolution matrix was applied to the images in order to evaluate only the red staining. Afterwards the color space of the images was transformed from RGB into L*a*b* images and each pixel of the picture was evaluated according to its L* values. The obtained result was transformed in ranks of L* and then normalized on the selected area and the total amount of collagen determined using the equation: total collagen=(L*threshold−L*measured)/pixel. Two slides of each skin samples were processed by image acquisition and related analysis (i.e., 12 images for each treatment).

The amount of collagen III (“COL3”) expression was evaluated in the skin samples. Specifically, twelve skin samples were immunostained with antibodies. The papillary dermis was used for this analysis, being the papillary dermis is typically more subjected to collagen variation in response to application of personal care compositions. The selected area was carefully chosen in order to avoid the risk of including irregularities and agglomerates, such as blood vessels, sebaceous glands, or hair follicles. The evaluation was performed by estimating both color intensity and distribution with IMAGE J (NIH) analysis software. Semiquanitative evaluation of dermal marker was obtained.

All quantitative data was summarized in terms of the mean score for the application of each personal care composition. The measures of variation as standard deviation and standard error of mean (sem) was applied to the original scores. Differences between groups were evaluated by One-way anova with permutation test followed by Tukey's and t test with permutation.

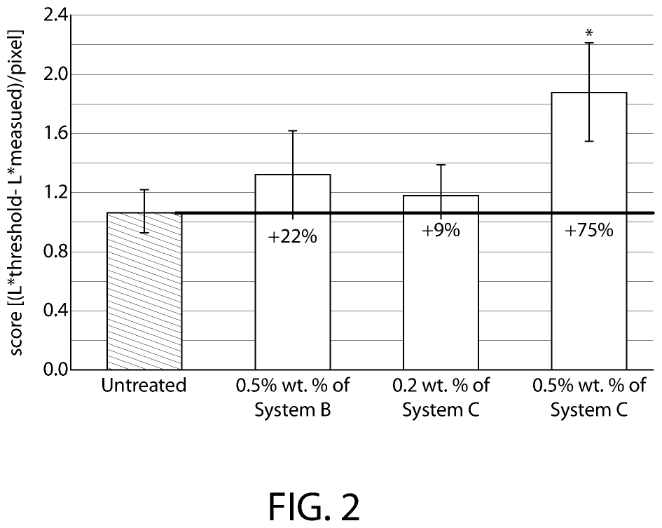

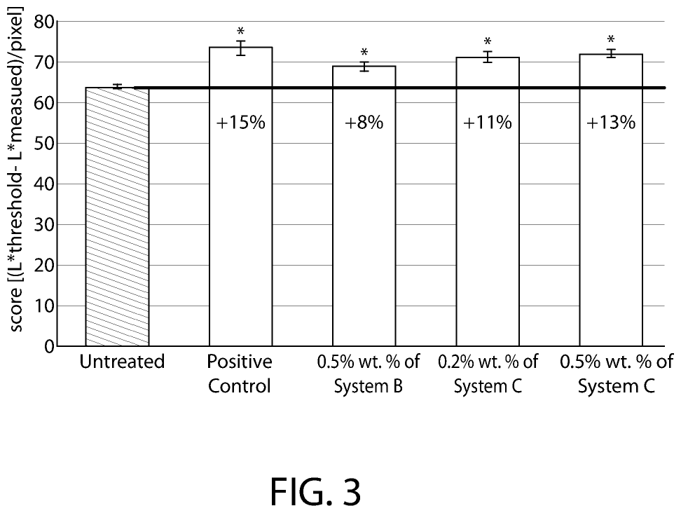

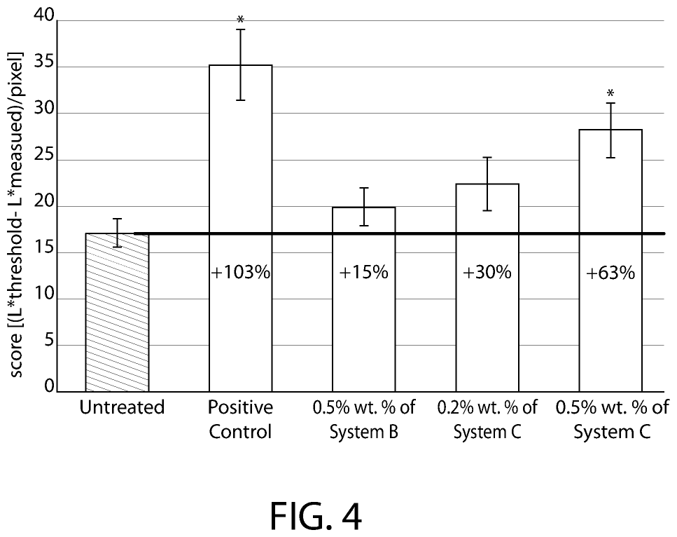

The amount of Filaggrin expression for the skin samples is shown in FIG. 2. The total amount of collagen expression for the skin samples is shown in FIG. 3. The amount of collagen III expression for the skin samples is shown in FIG. 4.

### Example 4

Three non-limiting, example compositions (Example Composition D-F) may be prepared in the form of a serum by incorporating Systems A-C from Table 1 in accordance with formulations shown in Table 2.

TABLE 2

| | Ex. D | Ex. E | Ex. F |
|---|---|---|---|
| INCI Compounds | (wt. %) | (wt. %) | (wt. %) |
| Water | QS to 100 | QS to 100 | QS to 100 |
| 1,3 Butanediol | 5 | 5 | 5 |
| Xanthan | 0.6 | 0.6 | 0.6 |
| PEG-40 | 3 | 3 | 3 |
| Hydrogenated Castor | | | |
| Oil | | | |
| Caprylic/Capric | 0.8 | 0.8 | 0.8 |
| Triglyceride | | | |
| Caprylyl Glycol | 0.3 | 0.3 | 0.3 |
| Sodium Benzoate | 0.5 | 0.5 | 0.5 |
| Purox S Grains | | | |
| Potassium Sorbate | 0.2 | 0.2 | 0.2 |
| System A | 0.1 to 0.9 | | |
| System B | | 0.1 to 0.9 | |
| System C | | | 0.1 to 0.9 |

1-32. (canceled)

33. A post-biotic composition comprising:
(a) a short chain fatty acid blend comprising:
a first short chain fatty acid selected from lactic acid, salt thereof, and a combination thereof, and
(b) a plurality of amino acids, the plurality of amino acids comprising at least 2 amino acids selected from histidine, glycine, threonine, arginine, alanine, valine, proline, a salt thereof, and a combination of two or more thereof.

34. The post-biotic composition according to claim 33, wherein the first short chain fatty acid and/or a salt thereof is present in an amount from about 50 to about 90 wt. %, based on the total weight of the post-biotic composition.

35. The post-biotic composition according to claim 33, wherein the short chain fatty acid blend further comprises:
a second short chain fatty acid selected from acetic acid, pyruvic acid, a salt thereof, and a combination of two or more thereof.

36. The post-biotic composition according to claim 35, wherein the second short chain fatty acid is pyruvic acid, sodium pyruvate, or a combination thereof.

37. The post-biotic composition according to claim 35, wherein the total amount of the first short chain fatty acid and/or a salt thereof to the total amount of the second short chain fatty acid and/or a salt thereof is from about 9:1 to about 2:1.

38. The post-biotic composition according to claim 35, wherein the second short chain fatty acid and/or a salt thereof is present in an amount from about 1 to about 50 wt. %, based on the total weight of the post-biotic composition.

39. The post-biotic composition according to claim 33, wherein the plurality of amino acids comprises at least 4 amino acids selected from histidine, glycine, threonine, arginine, alanine, valine, proline, a salt thereof, and a combination of two or more thereof.

40. The post-biotic composition according to claim 33, wherein the plurality of amino acids comprises at least 6 amino acids selected from histidine, glycine, threonine, arginine, alanine, valine, proline, a salt thereof, and a combination of two or more thereof.

41. The post-biotic composition according to claim 33, wherein the plurality of amino acids comprises of glycine, arginine, and alanine.

42. The post-biotic composition according to 41, wherein at least 50 wt. % of the plurality of amino acids is comprised of glycine, arginine, and alanine, based on the total weight of the plurality of amino acids.

43. A personal care composition comprising:
a polyol;
optionally, a fatty compound;
optionally, a thickener; and
a post-biotic system comprising:
(a) a short chain fatty acid blend comprising:
(i) a first short chain fatty acid selected from lactic acid, salt thereof and a combination thereof, and
(ii) a second short chain fatty acid selected from acetic acid, pyruvic acid, a salt thereof, and a combination of two or more thereof, and
(b) a plurality of amino acids, the plurality of amino acids comprising at least 2 amino acids selected from histidine, glycine, threonine, arginine, alanine, valine, proline, a salt thereof, and a combination of two or more thereof.

44. The personal care composition according to claim 43 further comprising:
alpha hydroxy acid(s) and/or a salt(s) thereof, optionally, in an amount from about 0.1 to about 6 wt. %, based on the total weight of the personal care composition.

45. The personal care composition according to claim 43 further comprising:
beta hydroxy acid(s) and/or a salt(s) thereof, optionally, in an amount from about 0.05 to about 2 wt. %, based on the total weight of the personal care composition.

46. The personal care composition according to claim 43 further comprising:
polyhydroxy acid(s) and/or a salt(s) thereof, optionally, in an amount from about 0.1 to about 5 wt. %, based on the total weight of the personal care composition.

47. The personal care composition according to claim 43, wherein the polyol is a humectant selected from triethylene glycol, tripropylene glycol, propylene glycol, glycerin, polyethylene glycols, polypropylene glycol, glycerin, sorbitol, hexylene glycol, butylene glycol, hyaluronic acid, 1,2 hexanediol, saccharide isomerate, and a combination of two or more thereof.

48. The personal care composition according to claim 43 comprising a fatty compound in an amount from about 0.1 to about 20 wt. %, based on the total weight of the personal care composition.

49. The personal care composition according to claim 43, wherein the total amount of the first short chain fatty acid and/or a salt thereof to the total amount of the second short chain fatty acid and/or a salt thereof is from about 9:1 to about 2:1.

50. The personal care composition according to claim 43, wherein the plurality of amino acids comprises at least 4 amino acids selected from histidine, glycine, threonine, arginine, alanine, valine, proline, a salt thereof, and a combination of two or more thereof.

51. The personal care composition according to claim 43, wherein the plurality of amino acids comprises at least 6 amino acids selected from histidine, glycine, threonine, arginine, alanine, valine, proline, a salt thereof, and a combination of two or more thereof.

52. The personal care composition according to claim 43, wherein the plurality of amino acids comprises of glycine, arginine, and alanine.
